(19) United States
(12) Patent Application Publication
(10) Pub. No.: US 2024/0389695 A1
(43) Pub. Date: Nov. 28, 2024

(54) Fertility Treatment Compression Belt Kit

(71) Applicant: Dandi Fertility, Inc., New York, NY (US)

(72) Inventors: Marni Katz Zimmerman, New York, NY (US); Jacob Kent, New York, NY (US)

(21) Appl. No.: 18/670,363

(22) Filed: May 21, 2024

Related U.S. Application Data
(60) Provisional application No. 63/468,105, filed on May 22, 2023.

(51) Int. Cl. A41F 9/02

(52) U.S. Cl. CPC A41F 9/025

(57) ABSTRACT

A Fertility Treatment Compression Belt Kit includes a flexible, stretchable belt with a cutout supported by a rigid frame that holds accessories such as heating and cooling pads. The belt features temporary tattoos to mark injection sites and a massage ball for use post-injection. The heating pad includes adjustable temperature settings and a pocket for warming medication vials, while the cooling pad provides pre-injection numbing. The temporary tattoo indicates the injection location, ensuring consistent application and belt alignment. The methods of use include applying the belt and inserting the cooling pad into the belt to numb the injection site, administering subcutaneous or intramuscular injections through the belt's cutout, inserting the heating pad into the belt to apply heat therapy, and using the massage ball at the injection site for post-injection therapy. The heating pad's pocket allows for pre-warming medication vials.

### CROSS-REFERENCE TO RELATED APPLICATIONS

This application claims the benefit of U.S. Provisional Application No. 63/468, 105, filed May 22, 2023, which is hereby incorporated by reference.

### BACKGROUND OF THE INVENTION

In vitro fertilization (IVF) is a widely recognized and utilized method for assisting individuals and couples in the conception of a child. The IVF process involves a complex series of protocols, including the preparation and administration of various fertility medications. These medications are typically self-administered through subcutaneous or intramuscular injections over an extended period, often spanning several days or weeks. The purpose of these medications is to stimulate the ovaries, prepare the patient's body for the retrieval of eggs, and facilitate the transfer of embryos.

Despite the advancements in reproductive medicine, the process of administering IVF injections remains a significant challenge for many patients. The current state of the art lacks products specifically designed to provide comfort, reduce pain, and simplify the injection process for those undergoing IVF treatment. The administration of fertility medications involves precise timing and dosage, which can be daunting and confusing, especially for individuals with limited medical experience.

Patients often report experiencing discomfort, anxiety, and pain associated with the frequent injections required during IVF cycles. This can lead to improper administration of medication, increased stress levels, and a negative overall experience. The repetitive nature of the injections, coupled with the emotional and physical strain of the IVF process, underscores the necessity for a solution that addresses these issues comprehensively.

Currently, there is a conspicuous absence of individual products tailored to enhance the comfort and ease of self-administering IVF injections. Existing solutions do not adequately cater to the unique needs of IVF patients, who require not only pain relief but also intuitive guidance to ensure accurate and effective medication delivery. The lack of such products can result in suboptimal treatment outcomes and heightened patient distress.

Therefore, there is a clear and pressing need in the field of reproductive medicine to develop innovative products that provide holistic support during the IVF injection process. Such products should aim to minimize pain, enhance patient comfort, and offer intuitive, user-friendly features to guide patients through the medication administration protocol. Addressing these needs will significantly improve the IVF experience, potentially leading to better adherence to treatment regimens and improved clinical outcomes. Thus, there is a need for a fertility treatment compression belt kit.

### SUMMARY OF THE INVENTION

A fertility treatment compression belt kit comprises a compression belt. The compression belt comprises a cutout and has a top side, bottom side, left side, right side, back side, and front side. The cutout exposes an area of the user's skin when the belt is worn. The compression belt is stretchable. A rigid frame is affixed to the compression belt and surrounds the cutout to provide support for an accessory. A connector is affixed to the right side of the belt and a connector is affixed to the left side of the belt. In one embodiment the connectors include a hook and loop fastener.

The kit further comprises an accessory having a top side, a bottom side, and a top cap affixed to the top side. The top side and bottom side of the accessory are larger than the cutout of the belt. The top cap extends from the top side of the accessory, and the top cap is congruent to the cutout of the belt. When the accessory is inserted into the compression belt, the top cap extends upwardly from the bottom side of the belt, through the cutout, out through the top side of the compression belt. In one embodiment the accessory is a cooling pad. In another embodiment the accessory is a heating pad. The heating pad includes a medication vial heating pocket.

The kit further comprises a temporary tattoo injection site shot target, wherein the shot target is smaller than the size of the cutout. The kit additionally includes a belt extender. The kit also includes a massage ball.

A method for performing fertility treatments comprises applying the fertility treatment compression belt so the cutout of the belt is aligned with an injection site of a user. The cooling pad is inserted into the cutout to numb the injection site. When the injection site is numb, the cooling pad is removed from the cutout of the belt. Medication is injected through the cutout. In one embodiment, prior to injecting the medication, the medication vial containing the medication is warmed with the heating pad by placing it in the pocket of the heating pad. After the injection is administered, a heating pad is inserted into the cutout to soothe the site.

The method may further comprise applying a temporary tattoo shot target to the injection site, and aligning the cutout of the belt with the temporary tattoo. Additionally, the method may comprise removing the heating pad and the belt, and massaging the injection site with a massage ball.

### BRIEF DESCRIPTION OF THE DRAWINGS

FIG. 1 is a Top Front Right Side Isometric view a Fertility Treatment Compression Belt in an open position.

FIG. 2 is a Bottom Rear Left Side Isometric view thereof.

FIG. 3 is a Front Elevation view thereof.

FIG. 4 is a Rear Elevation view thereof.

FIG. 5 is a Left Side Elevation view thereof.

FIG. 6 is a Right Side Elevation view thereof.

FIG. 7 is a Top Plan view thereof.

FIG. 8 is a Bottom Plan view thereof.

FIG. 9 is a Top Front Right Side Isometric view of the Fertility Treatment Compression Belt of FIG. 1, with the belt in a closed position.

FIG. 10 is a Bottom Rear Left Side Isometric view thereof.

FIG. 11 is a Front Elevation view thereof.

FIG. 12 is a Rear Elevation view thereof.

FIG. 13 is a Left Side Elevation view thereof.

FIG. 14 is a Right Side Elevation view thereof.

FIG. 15 Top Plan view thereof.

FIG. 16 is a Bottom plan view thereof.

FIG. 17 is an exploded view of the Fertility Treatment Compression Belt.

FIG. 18 is a Top Front Right Side Isometric view of a Belt Extender.

FIG. 19 is a Bottom Rear Left Side Isometric view thereof.

FIG. 20 is a Front Elevation view thereof.

FIG. 21 is a Rear Elevation view thereof.

FIG. 22 is a Left Side Elevation view thereof.

FIG. 23 is a Right Side Elevation view thereof.

FIG. 24 is a Top Plan view thereof.

FIG. 25 is a Bottom Plan view thereof.

FIG. 26 is a Top Front Right Side Isometric view of a Cooling Pad.

FIG. 27 is a Bottom Rear Left Side Isometric view thereof.

FIG. 28 is a Front Elevation view thereof.

FIG. 29 is a Rear Elevation view thereof.

FIG. 30 is a Left Side Elevation view thereof.

FIG. 31 is a Right Side Elevation view thereof.

FIG. 32 is a Top Plan view thereof.

FIG. 33 is a Bottom Plan view thereof.

FIG. 34 is a Top Front Right Side Isometric view of a Heating Pad.

FIG. 35 is a Bottom Rear Left Side Isometric view thereof.

FIG. 36 is a Front Elevation view thereof.

FIG. 37 is a Rear Elevation view thereof.

FIG. 38 is a Left Side Elevation view thereof.

FIG. 39 is a Right Side Elevation view thereof.

FIG. 40 is a Top Plan view thereof.

FIG. 41 is a Bottom Plan view thereof.

FIG. 42 is a Top Front Right Side Isometric view of a Massage Ball.

FIG. 43 is a Bottom Rear Left Side Isometric view thereof.

FIG. 44 is a Front Elevation view thereof.

FIG. 45 is a Rear Elevation view thereof.

FIG. 46 is a Left Side Elevation view thereof.

FIG. 47 is a Right Side Elevation view thereof.

FIG. 48 is a Top Plan view thereof.

FIG. 49 is a Bottom Plan view thereof.

FIG. 50 is a Top Front Right Side Isometric view of a Temporary Tattoo Injection Site Shot Target.

FIG. 51 is a Bottom Rear Left Side Isometric view thereof.

FIG. 52 is a Front Elevation view thereof.

FIG. 53 is a Rear Elevation view thereof.

FIG. 54 is a Left Side Elevation view thereof.

FIG. 55 is a Right Side Elevation view thereof.

FIG. 56 is a Top Plan view thereof.

FIG. 57 is a Bottom Plan view thereof.

FIG. 58 is a method for performing fertility treatments.

### DETAILED DESCRIPTION

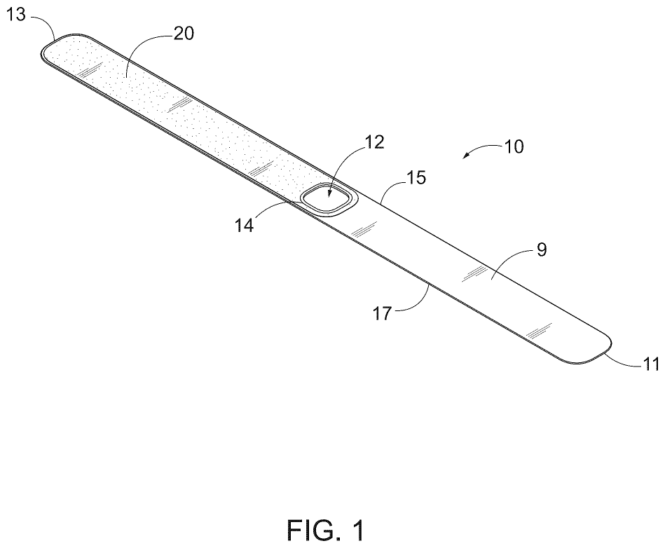

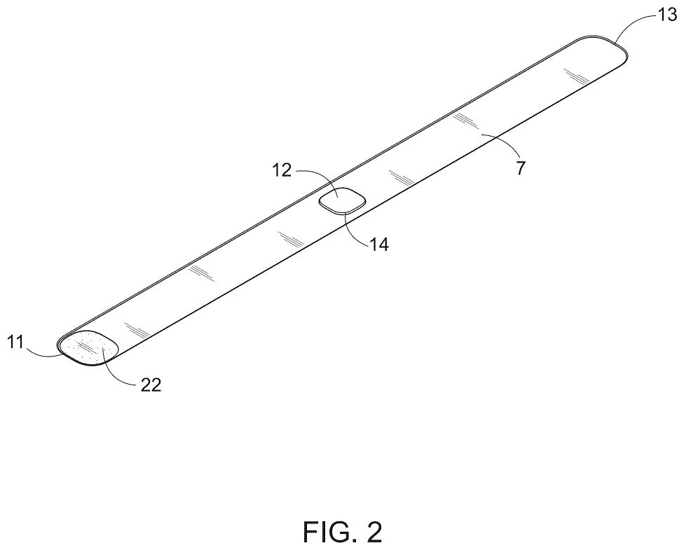

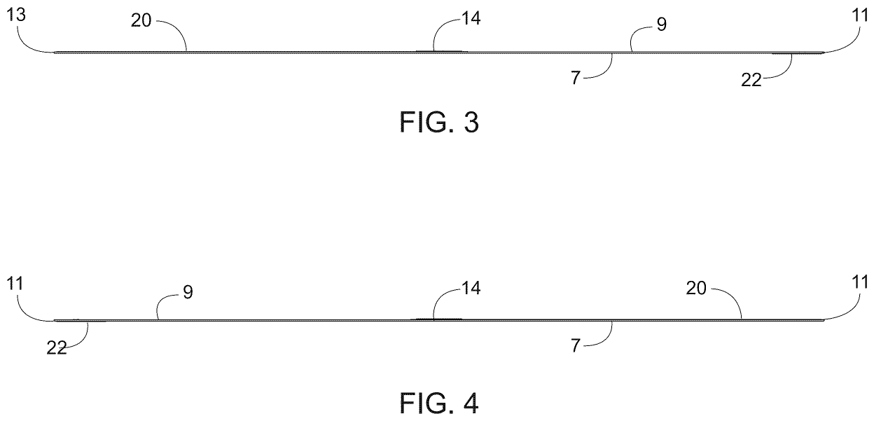

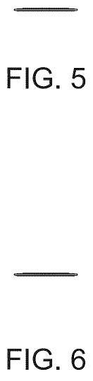

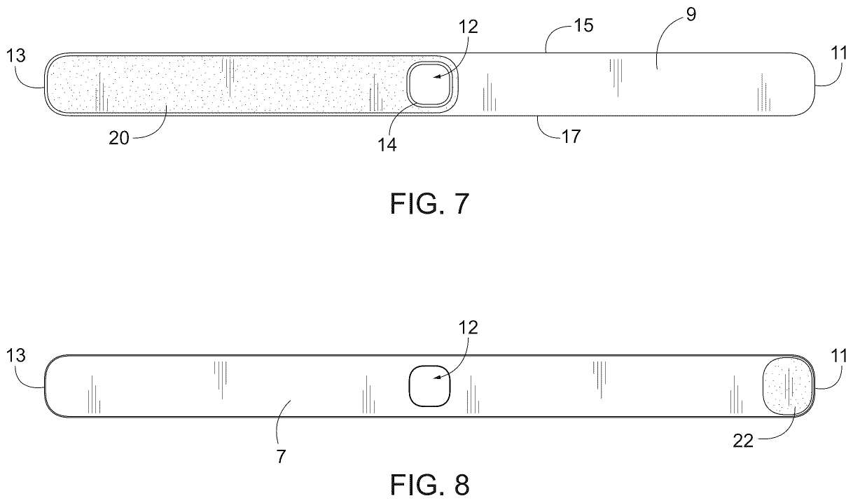

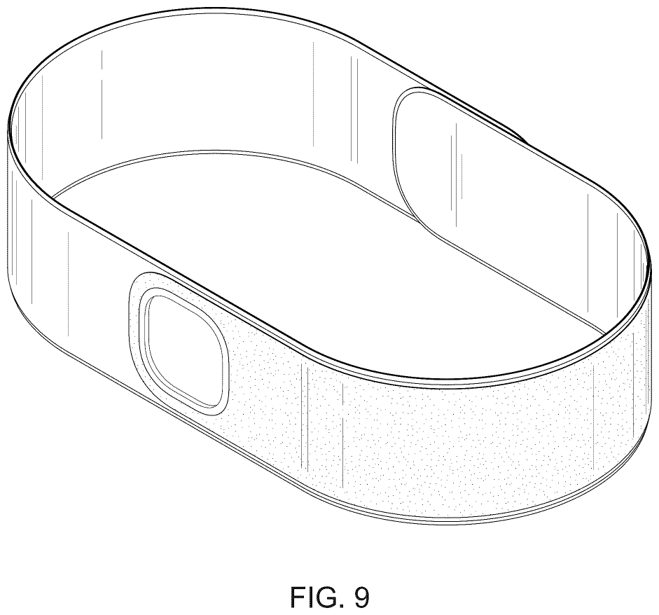

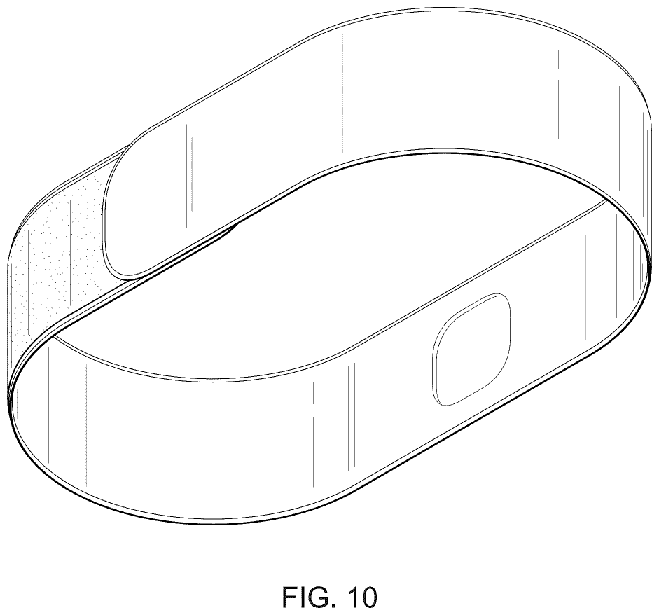

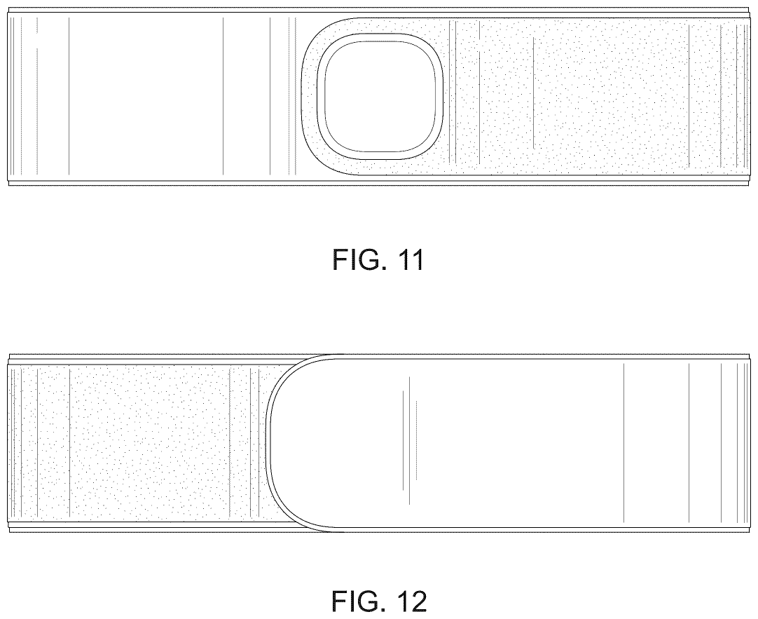

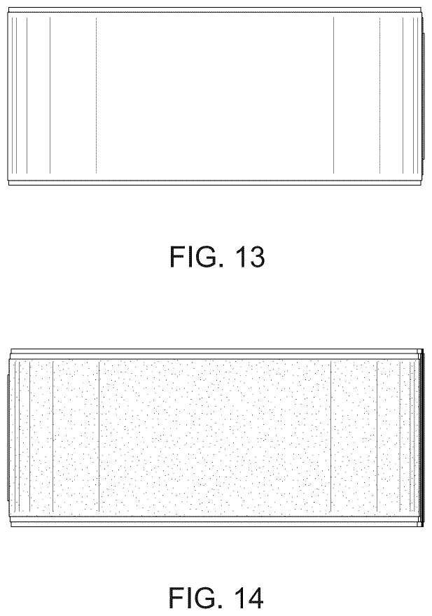

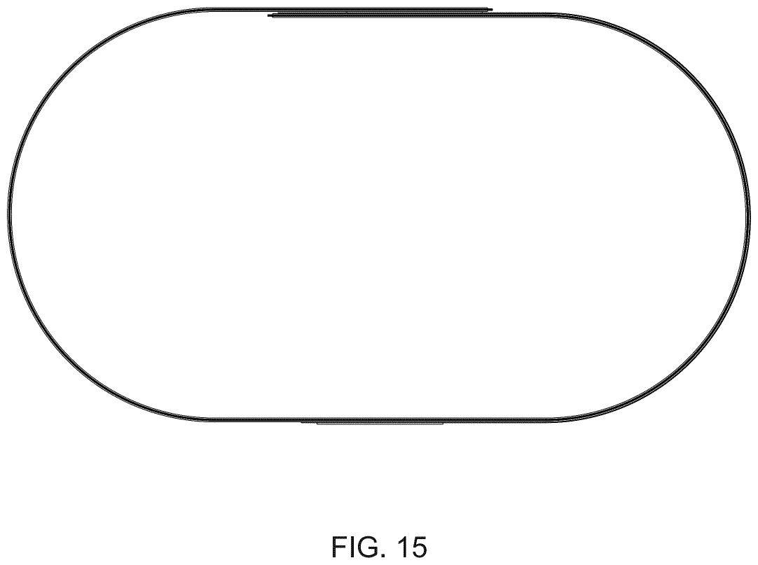

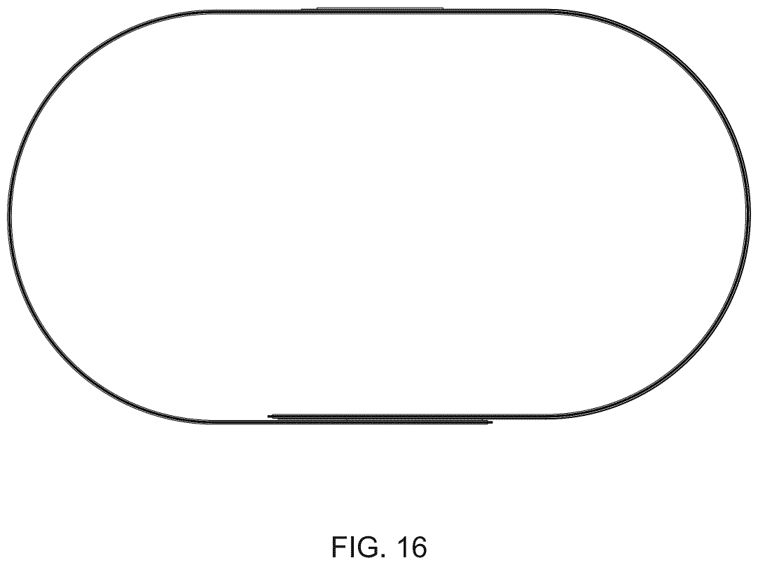

FIGS. 1-8 depict a Fertility Treatment Compression Belt (hereinafter referred to as the “Belt”) 10 in an open position, while FIGS. 9-16 illustrate the Belt 10 in a closed position. The Belt is designed to provide comfort and support to users undergoing fertility treatments, particularly during the administration of injections. The Belt 10 features a top side 9, a bottom side 7, a left side 13, a back side 15, and a front side 17, ensuring comprehensive coverage and support.

A notable feature of the Belt is the cutout 12, also referred to as an accessory cutout. This cutout 12 forms an opening, or window, through Belt 10, allowing direct access to the user's skin for the administration of injections when the Belt is worn. Cutout 12 is specifically designed to accommodate and securely hold an accessory, such as a heating pad or cooling pad, which will be discussed in further detail later.

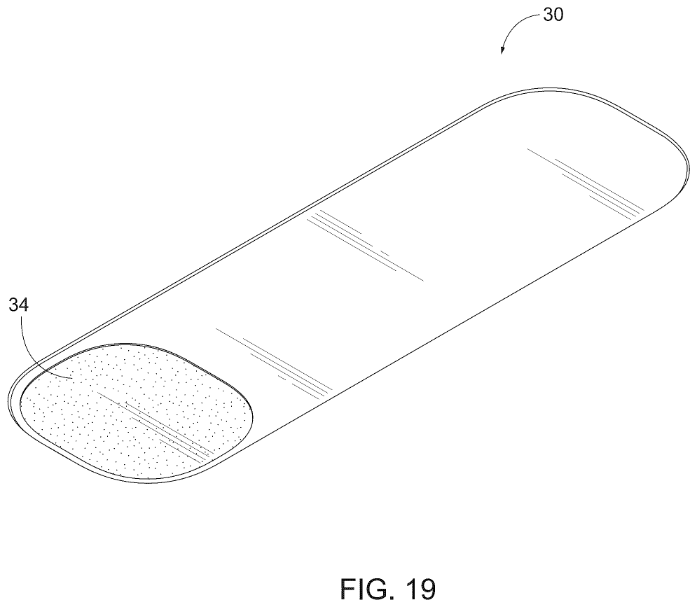

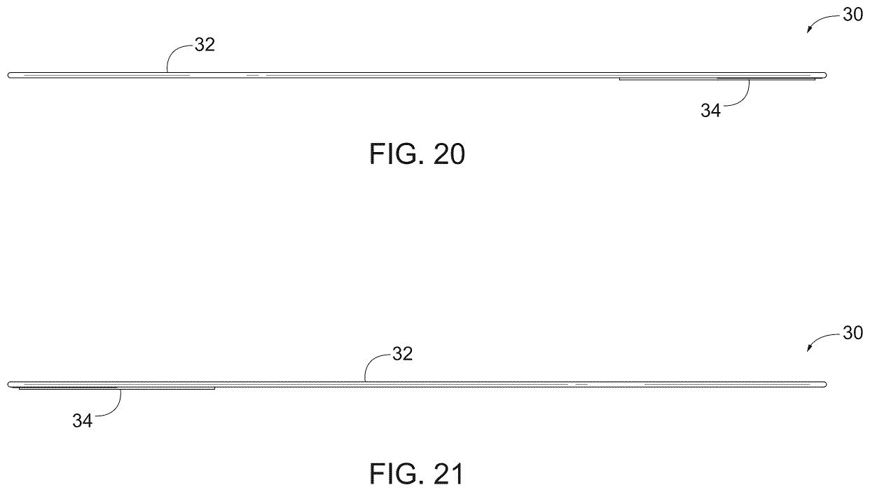

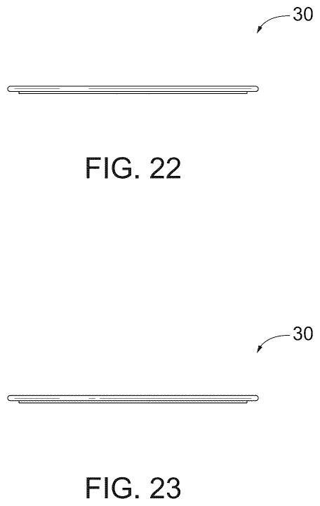

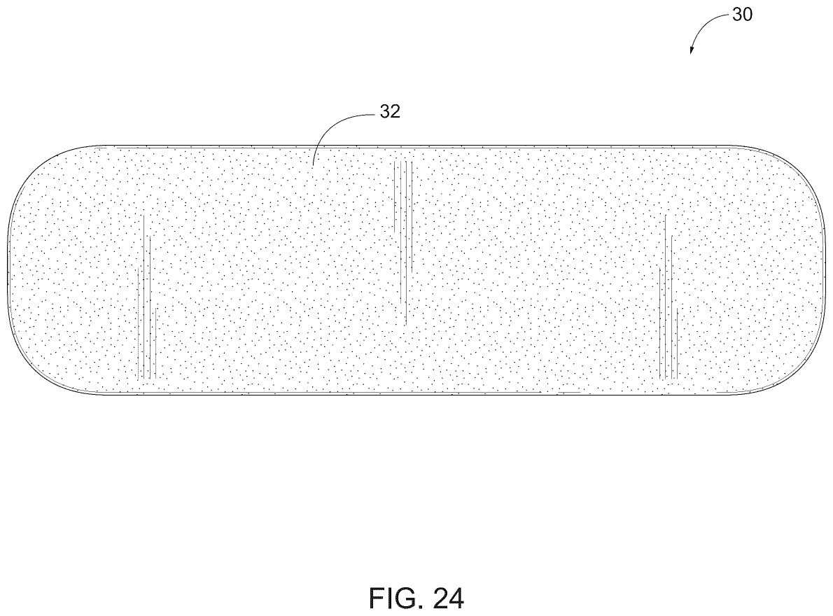

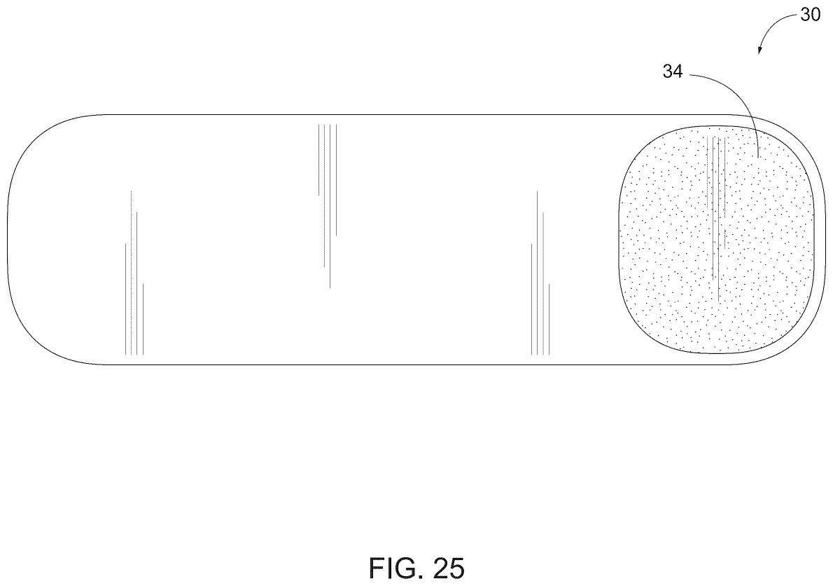

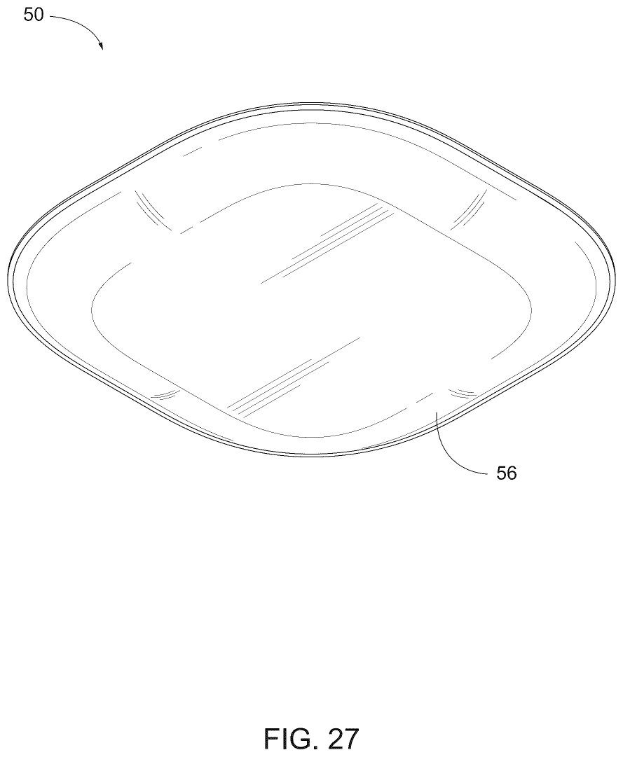

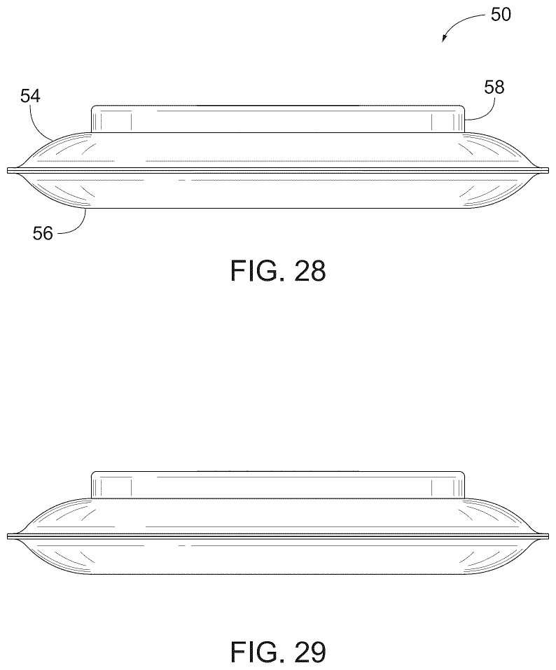

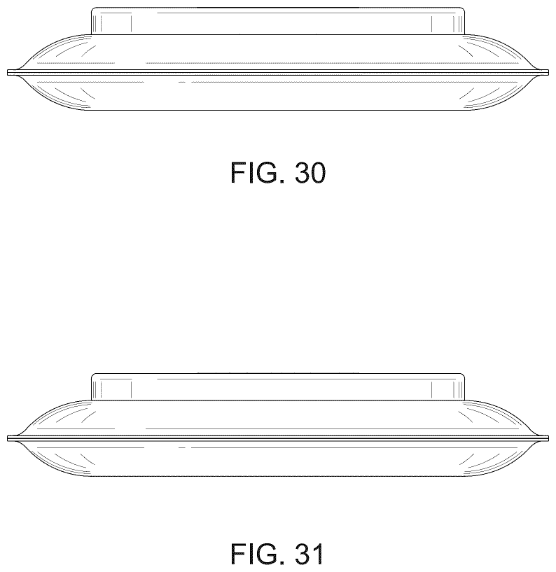

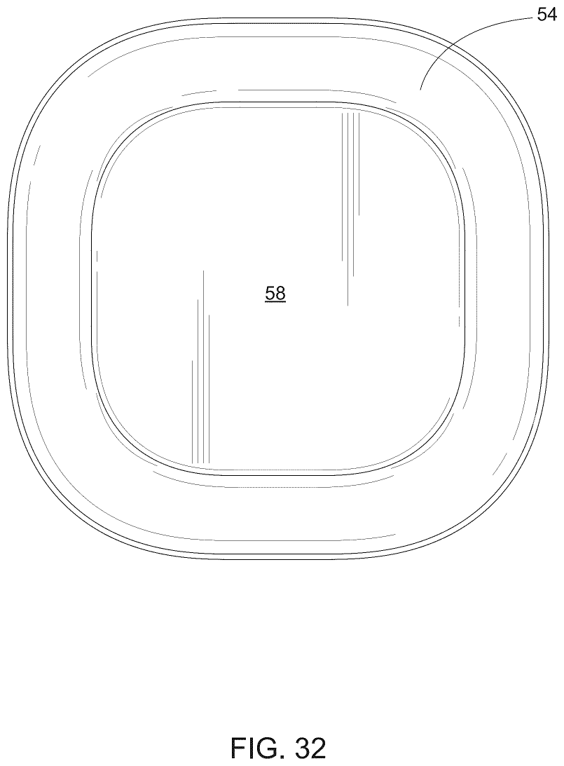

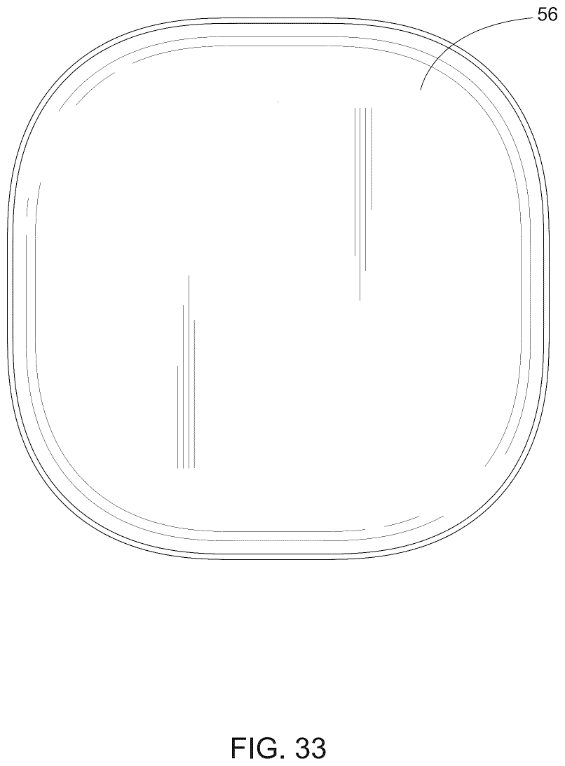

Surrounding and enclosing cutout 12 is a rigid frame 14, which provides structural support and stabilization. In one embodiment, the rigid frame 14 is constructed from durable plastic, ensuring both strength and lightness. As detailed in FIGS. 18-33, when a specially designed accessory, such as a heating pad or cooling pad, is inserted into cutout 12, the frame 14 offers support, maintaining the accessory in a stable and fixed position while the Belt 10 is worn.

The rigid frame 14 helps ensure the effective use of the accessory. When the Belt is worn snugly around the user's body, frame 14 applies consistent pressure around the accessory. This pressure ensures that the accessory maintains firm contact with the user's skin, enhancing its effectiveness, whether it is providing therapeutic heat or cold. Frame 14 also prevents the accessory from shifting or moving, which could otherwise lead to discomfort or reduced efficacy.

This design not only improves the comfort and usability of the Belt 10 but also enhances the overall experience for the user by ensuring that the accessory remains securely in place, maximizing its therapeutic benefits. The strategic placement of the cutout 12, combined with the supportive frame 14, makes the Belt an innovative solution for users undergoing fertility treatments, providing them with both comfort and functionality.

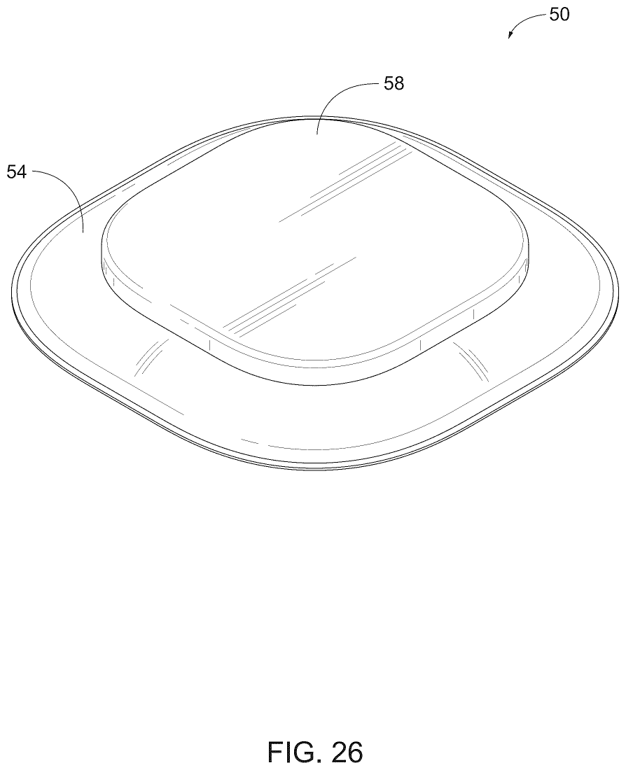

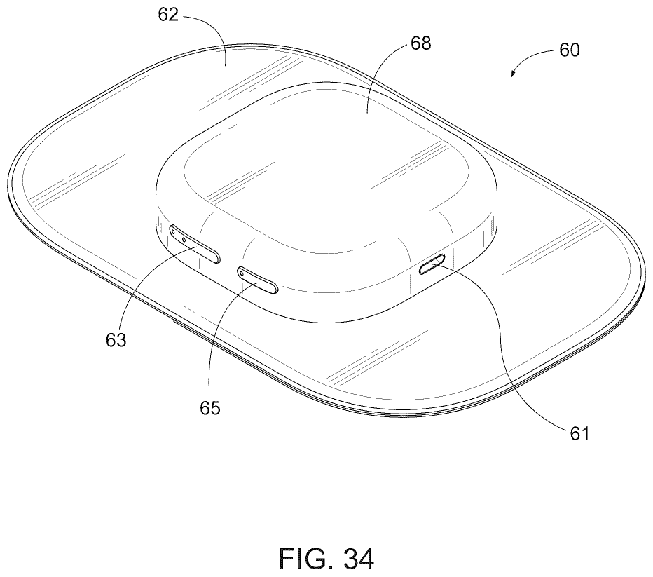

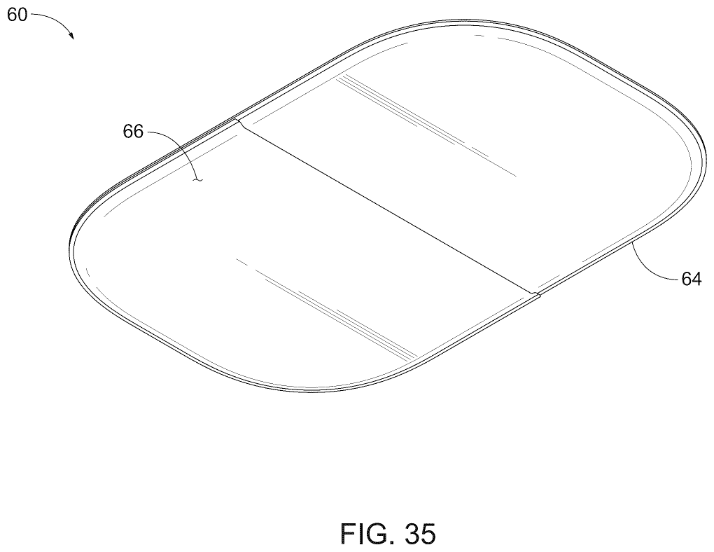

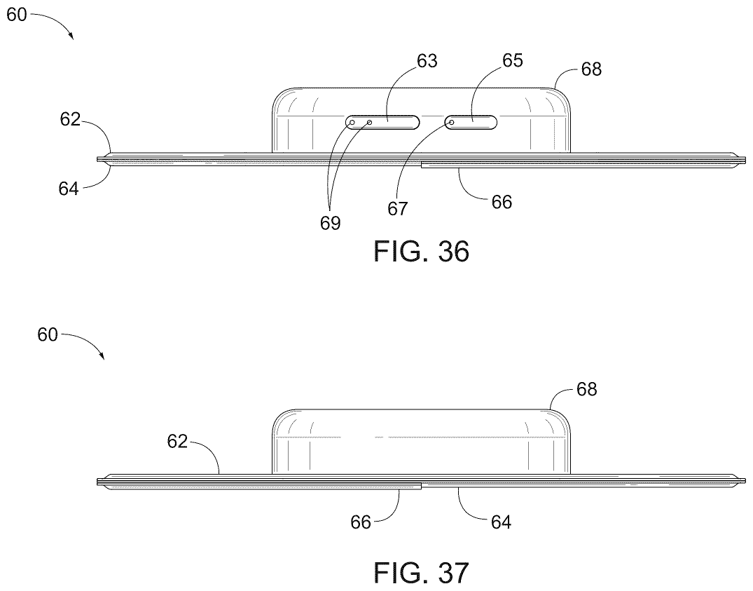

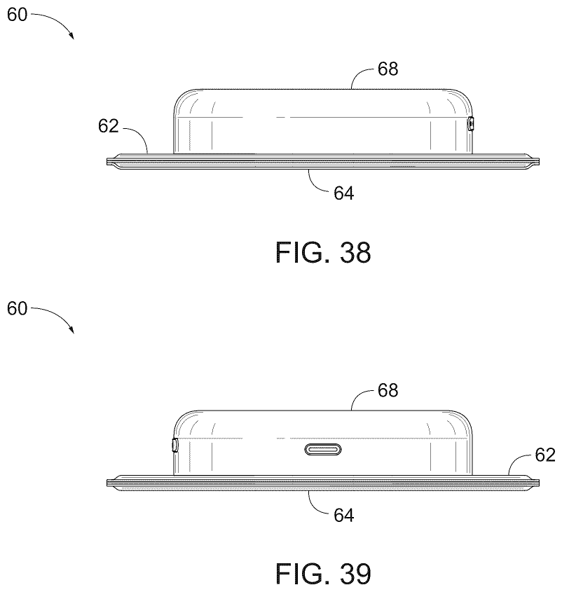

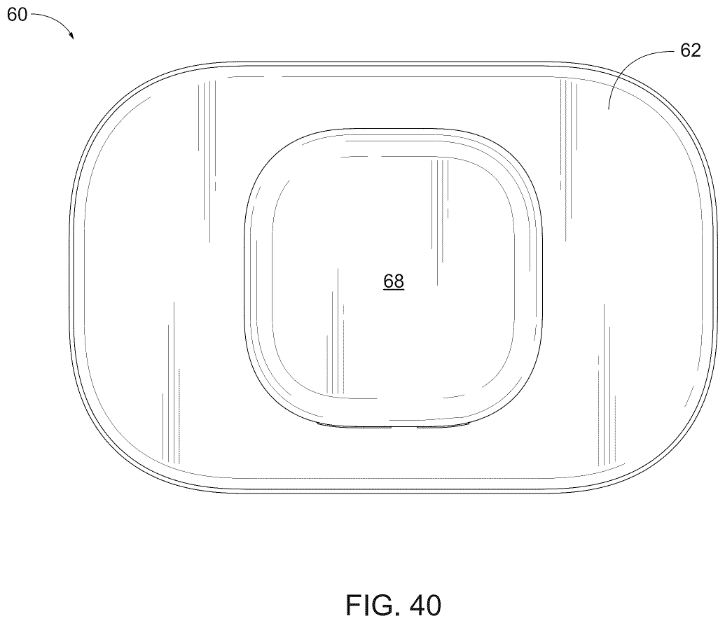

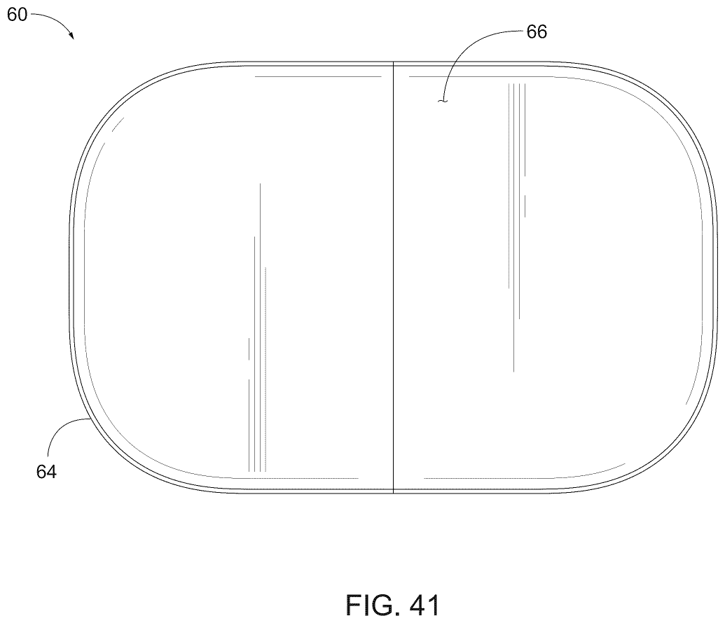

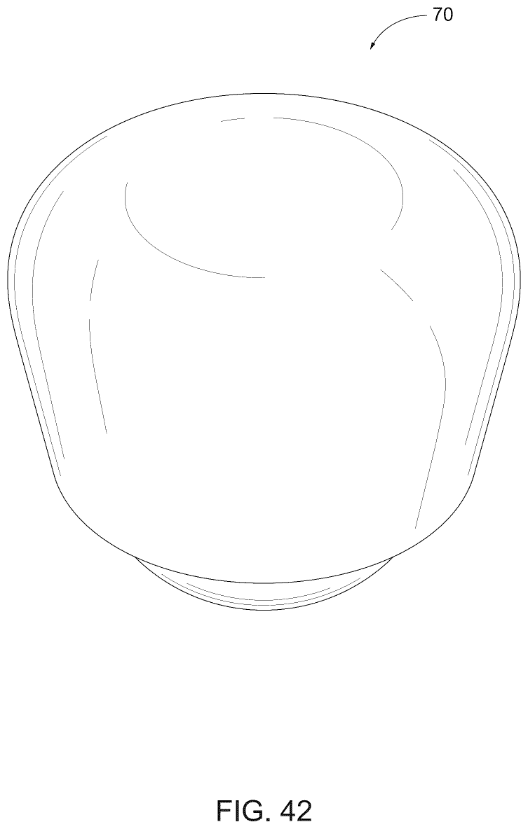

The shape of cutout 12 and the inner perimeter of frame 14 are designed to be congruent, ensuring a clean, smooth appearance and facilitating the easy and smooth insertion and removal of the accessory. This design choice ensures that the accessory fits seamlessly into the frame, enhancing both the functionality and aesthetic appeal of the Belt. As will be detailed later with reference to the cooling pad (FIGS. 26-33) and heating pad (FIGS. 34-41), the accessory features a matching raised cap that fits into and extends through frame 14, thereby being securely and stably held in place. This secure fit maximizes the therapeutic benefits of the accessories by maintaining optimal contact with the user's skin even while the user is moving around.

In the embodiment illustrated in the figures, cutout 12 and the inner perimeter of frame 14 are shaped as a rounded quadrilateral. However, this disclosure is not limited to such a shape. Other shapes are possible, including but not limited to polygons, rounded polygons, circles, and various other straight-sided and curved shapes. The key is that the cutout and frame are designed to match in shape, ensuring the accessory can be securely and effectively held in place regardless of the specific geometric configuration.

Belt 10 is constructed to be flexible and stretchable, providing both comfort and functionality. It is made from one or more types of materials that can stretch or expand and then return to their original shape. Examples of such materials include but are not limited to elastane, polychloroprene, stretch cotton, stretch denim, stretch knits, stretch velvet, and stretch mesh. These stretchable materials may be combined with other natural and synthetic materials such as but not limited to cotton, linen, wool, silk, hemp, bamboo, polyester, nylon, acrylic, viscose, and polypropylene. The choice of materials ensures that the Belt 10 can offer varying degrees of elasticity depending on the specific embodiment.

Additionally, the materials may be selected for their breathability, water resistance, and stain resistance properties to enhance user comfort and durability. For improved water and stain resistance, the materials may be treated with coatings such as, but not limited to, the products sold under the trademarks TEFLON, SCOTCHGARD, or silicone-based finishes. Durable water repellent (DWR) treatments can also be applied to fabrics to enhance their resistance to moisture. These treatments and coatings help protect the Belt from water and stains, ensuring it remains functional and aesthetically pleasing over time. The combination of breathability and resistance properties makes Belt 10 suitable for extended wear, providing users with both comfort and practical benefits.

Knit textiles used in the construction of Belt 10 can have two-way or four-way stretch properties, allowing for a more adaptable and comfortable fit. The combination of different materials aims to achieve specific characteristics that enhance the overall user experience, ensuring Belt 10 is soft, comfortable, suitable for prolonged wear, and durable.

The materials and construction methods used in the Belt 10 are chosen to provide a soft and comfortable experience for the user. This is crucial as the Belt is intended to be worn for extended periods, particularly during the administration of fertility treatments, which can be a stressful and physically demanding process. The careful selection of materials and design considerations ensures that the Belt 10 not only supports the user physically but also contributes to their comfort and well-being during a challenging time.

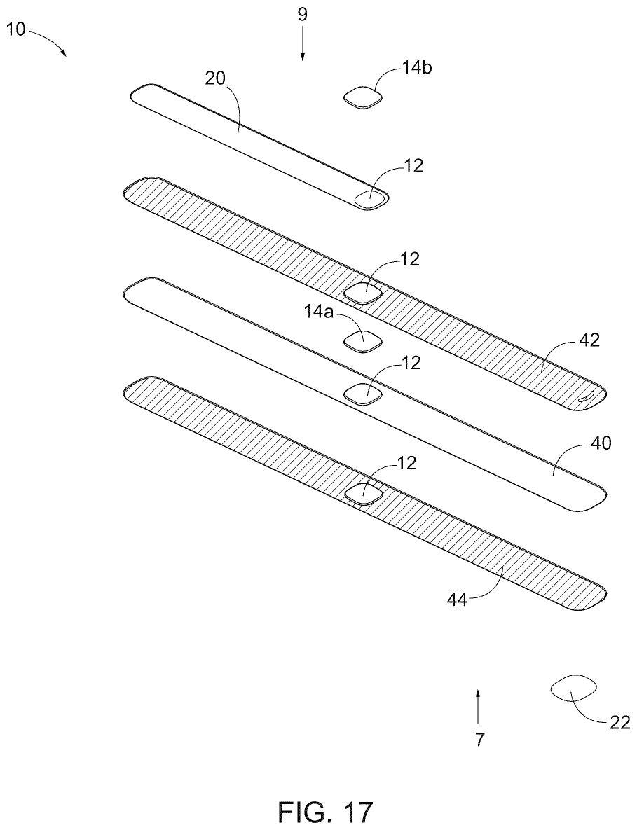

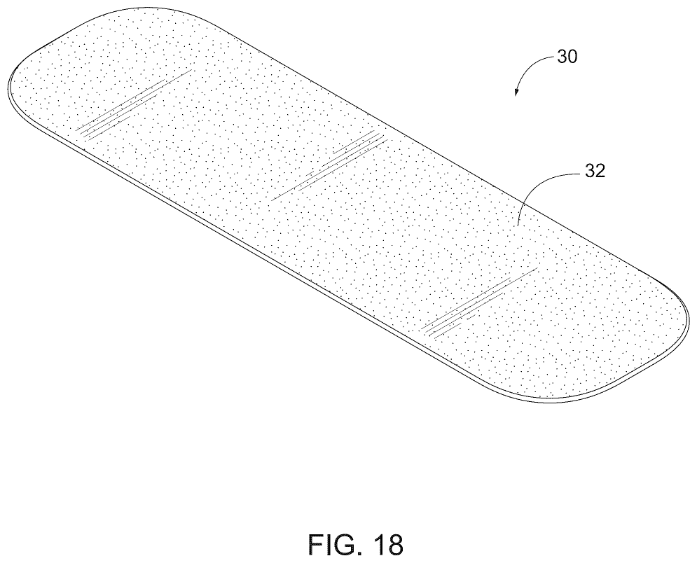

To secure the Belt 10 around a user, the Belt includes connectors 20 and 22. Connector 22 is affixed to the bottom right side 7 of the Belt 10, and connector 20 is affixed to the top left side 9 of the Belt 10. In one embodiment, connectors 20 and 22 comprise a hook and loop fastener. In one embodiment, connector 20 is the loop portion of a hook and loop laminate, and connector 22 is the hook portion of a hook and loop laminate. A common example of a hook and loop fastener is the product sold under the trademark VELCRO, although other hook and loop fasteners may also be used. The hook and loop fastener configuration shown in FIGS. 1-16 offers the significant advantage of being almost infinitely adjustable, allowing the user to precisely adjust the Belt 10 for optimal snugness and comfort. Additionally, hook and loop fasteners provide advantages during the construction and manufacture of the Belt 10, as they come in sheets and can be easily bonded to various fabrics. The construction of the Belt 10 will be disclosed later with reference to FIG. 17.

While hook and loop fastener are utilized in this embodiment for its ease of use and adjustability, it is understood by those skilled in the art that other types of fasteners can also be employed to close Belt 10, even if they may not provide the same level of convenience or adjustability. Examples of such fasteners or closure mechanisms include clips, buckles, clasps, snap buttons, zippers, magnetic closures, and ties. Each of these alternative fasteners can securely close the Belt around the user, but they may vary in terms of adjustability, ease of use, and overall comfort.

In one embodiment, the length of Belt 10, that is the distance from left side 13 to right side 15, is 51 inches. The width of Belt 10, that is the distance from front side 17 to back side 15, is 4.5 inches. The thickness of Belt 10, that is the distance from the top side 9 to the bottom side is between approximately 1/16″ to approximately ⅛″. The cutout 12 and frame 14 are approximately centered lengthwise and widthwise on the Belt. The cutout 12 and frame 14 are shaped as a rounded square with a width and height of 2.75 inches.

It should be noted that these dimensions represent just one embodiment. Other lengths, widths, dimensions of Belt 10 and positions and sizes of cutout 12 are possible, as well as sizes, placements, and types of connectors 20 and 22. The critical consideration is that the Belt should be designed to fit most adult women, accommodating a wide variety of body shapes and sizes. Therefore, the specified dimensions should not be construed as limiting, and other configurations should be considered equivalent within the context of this disclosure. It is understood that those having ordinary skill in the art, given the benefit of this disclosure, would recognize that various modifications in dimensions and placements can be made without departing from the scope and spirit of the invention.

Turning now to FIGS. 18-25, the fertility treatment compression belt kit includes a belt extender 30 (the “Extender”). While Belt 10 is dimensioned to fit most adult women, accommodating a wide variety of body shapes and sizes, Extender 30 when connected to Belt 10 provides additional length to accommodate use of the kit with girthier women. Extender 30 is of similar construction and materials as Belt 10 disclosed above. The top of Extender 30 comprises a connector 32 which connects to connector 22 of Belt 10. The bottom of Extender 30 comprises connector 34 which connects to connector 20 of Belt 10. In this way, the belt can be extended if necessary.

In one embodiment, the length of Extender 30 is 14 inches and the width matches that of Belt 10. Connectors 32 and 34 are hook and loop fasteners. In one embodiment, connector 32 is the loop portion and connector 34 is the hook portion. As disclosed above with reference to Belt 10 and its dimensions and connectors, the dimensions of Extender 30 and the types of connectors disclosed herein are intended to be exemplary rather than limiting. The construction and materials of Extender 30 are similar to those of Belt 10.

The purpose of the Extender 30 is to accommodate users of various body sizes, ensuring that the Belt can be comfortably worn by a wide range of individuals. Therefore, different lengths and widths of the Extender 30 may be utilized to provide a proper fit for users with diverse body shapes and sizes. The specified dimensions and connectors should not be viewed as restrictive, and other configurations and materials that achieve the same function are considered within the scope of this disclosure. It is understood that those skilled in the art, given the benefit of this disclosure, would recognize that modifications can be made without departing from the spirit and scope of the invention.

FIG. 17 shows an exploded view of the Fertility Treatment Compression Belt as depicted in FIGS. 1-16. The Belt Extender shown in FIGS. 18-25 is similarly constructed. The Belt comprises a foam core 40 sandwiched between a top fabric 42 and a bottom fabric 44. These layers are affixed together to form a unified structure. Affixed to the top fabric 42 is the loop connector laminate 20, and affixed to the bottom fabric 44 is the hook connector laminate 22. The rigid frame 14 is positioned between the top fabric 42 and the bottom fabric 44. All of these components, including the top fabric 42, foam core 40, and bottom fabric 44 feature cutout 12 as disclosed above. The bottom fabric 44 is separated from the rigid frame 14 by the foam core 40, providing cushioning and comfort for the wearer.

In one embodiment, the rigid frame 14 comprises two parts: a bottom frame part 14a and a window frame part 14b. These parts are assembled as shown in FIG. 17, with the window frame part 14b affixed to the bottom frame part 14a. The window frame part 14b extends through the top fabric 42 and loop connector laminate 20, and is approximately flush or slightly proud of the loop connector laminate 20. The bottom frame part 14a, window frame part 14b, top fabric 42, loop connector laminate 20, foam core 40, and bottom fabric 44 are all securely affixed together.

In one embodiment, frame 14, and bottom frame part 14a in the case where frame 14 is a two-part frame, is constructed from a thin piece of plastic approximately 1/32 inch thick. The dimensions of frame 14 are carefully designed to match cutout 12 of belt 10. In this embodiment, the inner diameter of frame 14 is 2.75″×2.75″, forming a rounded square shape that aligns perfectly with cutout 12. The frame width, extending from the inner diameter to the outer edge is approximately ⅜″. Consequently, the overall outer dimensions of frame 14 is 3.5″×3.5″. This integration ensures that frame 14 is securely affixed into place and the thin yet durable construction ensures that it provides sufficient support without adding unnecessary bulk or discomfort to the wearer.

The elements of the Belt, as shown in FIG. 17, can be affixed using various methods well-known in the art to ensure a durable and reliable construction. In one embodiment, the components are bonded using adhesives and heat pressing techniques. For instance, fabric-to-fabric and fabric-to-foam bonds can be achieved using specialized fabric glues such as, but not limited to, the product sold under the trademark 3M SUPER 77 spray adhesive permanent adhesive. This adhesive provides strong, flexible bonds suitable for wearable items.

Heat pressing is another effective method for bonding fabrics, foam, and plastic components. In another embodiment, heat-activated adhesives, such as thermoplastic polyurethane (TPU) films, can be used to create strong bonds when heat and pressure are applied. This method ensures a seamless integration of the materials, enhancing both the durability and comfort of the Belt.

Additionally, the rigid frame 14, which may be made of plastic or other rigid materials, can be affixed to the foam core 40 and fabrics using industrial adhesives like epoxy resins or cyanoacrylates, known for their strong bonding properties. For example, the product sold under the trademark LOCTITE EPOXY PLASTIC BONDER is well-suited for bonding plastic to fabric and foam, providing a robust and long-lasting connection.

The construction of Belt 10 involves a combination of bonding techniques to ensure all components are securely affixed. The use of adhesives, heat pressing, and appropriate bonding agents allows for the durable assembly of the foam core, fabrics, and rigid frame, ensuring the belt maintains its integrity and provides maximum comfort and functionality for the user.

The accessories which are inserted into cutout 12 of the belt include a cooling pad shown in FIGS. 26-33 and a heating pad shown in FIGS. 34-41.

Referring to FIGS. 26-33, a cooling pad 50 is illustrated. The cooling pad 50 comprises a top side fabric 54 and a bottom side fabric 56, which are bonded together to encase a cool gel pack. The construction of the cool gel pack is well-known and typically includes a sealed pouch made of flexible, durable plastic or vinyl filled with a gel that can be placed in a freezer and retains cold temperatures of a typical home freezer for extended periods.

The top side fabric 54 and bottom side fabric 56 are congruent and treated with a water-repellent coating to ensure durability and comfort during use, as previously disclosed. These fabrics are larger in dimensions than the cutout 12 and the rigid frame 14 of the Belt 10. This size difference ensures that when the cooling pad 50 is inserted into the Belt, it is securely retained between the bottom side 7 of the Belt and the user's body. In one embodiment, the top side 54 and bottom side 56 are 4 inches by 4 inches and the corners are rounded.

A top cap 58, made of a rigid material such as plastic, is affixed to the top side fabric 54 and extends outwardly. The top cap 58 is designed to be congruent with the cutout 12, meaning it has the same size and shape as the cutout's inner dimensions. In other words, the plane of the top cap 58 has the same size and shape in dimensions as the cutout 12. This congruence allows the top cap 58 to fit seamlessly within the cutout 12, creating a secure and matching connection when inserted into the Belt. In one embodiment, the top cap 58 is a rounded quadrilateral 2.6875″×2.6875 to match the rounded quadrilateral of the cutout 12, and the height of the top cap is 3/16″, thereby extending from the top fabric 54 by 3/16″.

The cooling pad 50 is inserted into cutout 12 from the inner side of Belt 10. Once in place, rigid frame 14 of Belt 10 holds the cooling pad 50 securely, with the top cap 58 fitting flush with or standing proud of the loop connector 20. This design ensures that the cooling pad 50 remains stable and in optimal contact with the user's skin, providing effective cooling relief. The secure fit and placement allows for consistent therapeutic benefits, even as the user moves around, ensuring comfort and efficacy during use.

Referring to FIGS. 34-41, a heating pad 60 is illustrated. The heating pad 60 comprises a top side fabric 62 and a bottom side fabric 64, which are bonded together to envelop a heating film.

The top side fabric 62 and bottom side fabric 64 are congruent and treated with a water-repellent coating to ensure durability and comfort during use, as previously disclosed. These fabrics are larger in dimensions than cutout 12 and rigid frame 14 of Belt 10. This size difference ensures that when heating pad 50 is inserted into the Belt, it is securely retained between the bottom side 7 of the Belt and the user's body. In one embodiment, the top side 62 and bottom side 64 is a rounded rectangle having a width of 6 inches and a height of 4 inches.

The bottom side 64 of the heating pad 60 includes a vial pocket 66. This pocket is formed by bonding a piece of vial fabric 68, smaller than the bottom side 64, to the bottom fabric 64 on three edges, leaving one edge open to create a pocket. A vial of medication, such as progesterone, can be placed in pocket 66 and heated by the heating pad 60 prior to injection. Warming the medication helps to reduce the pain and discomfort associated with injections. In one embodiment pocket 66 is 4 inches wide (the same width as bottom 64) and 3 inches high, making a 4 inch pocket opening across the approximate center of the bottom.

A top cap 68, made of a rigid material such as plastic, is affixed to the top side fabric 62 and extends outwardly. The top cap 68 is designed to be congruent with the cutout 12, meaning it has the same size and shape as the cutout's inner dimensions. This congruence allows the top cap 68 to fit seamlessly within the cutout 12, creating a secure and matching connection when inserted into the Belt. In one embodiment, the top cap 58 is a rounded quadrilateral to match the rounded quadrilateral 2.6875″×2.6875″ and extends from the top fabric 62 by ⅝″.

The heating pad 60 is inserted into the cutout 12 from the bottom side 7 of the Belt 10. Once in place, the rigid frame 14 of the Belt 10 holds the cooling pad 60 securely, with the top cap 68 standing proud of the loop connector 20. This design ensures that the heating pad 60 remains stable and in optimal contact with the user's skin, providing effective heating relief. The secure fit and placement allows for consistent therapeutic benefits, even as the user moves around, ensuring comfort and efficacy during use.

Heating pad 60 includes multiple control buttons 63, 65 to regulate its heat settings effectively. Specifically, the heating pad 60 features vial heat button 65, which allow the user to control the heat settings of the pad to warm a vial medication in pocket 66. When the vial heat button 65 is activated, an LED indicator light 67 illuminates to show the heating status. In one embodiment, when vial heat button 65 is activated, the heating pad heats up to approximately 99.6 degrees Fahrenheit and remains at that temperature for 15 minutes to warm a progesterone vial placed in pocket 66. After 15 minutes, heating pad 60 turns off.

In addition to vial heat button 65, the heating pad 60 also includes a dedicated heat button 63 designed for controlling the temperature specifically for post-injection heating of the user's skin where the injection was made. Activating heat button 63 causes another LED indicator light or lights 69 to illuminate, signaling that the post-injection heating function is active. In one embodiment there are two heat settings, a low setting and a high setting, activated by pressing heat button 63 once or twice. Depending on the setting, one or two indicator lights 69 illuminate to communicate to the user the heat setting. In one embodiment the low heat setting heats the heating pad to approximately 100 degrees to 110 degrees Fahrenheit, and the high heat setting heats the heating pad to approximately 125 degrees to 135 degrees Fahrenheit. These temperatures are exemplary and should not be construed as limiting the invention. Other temperature ranges may be used depending on specific therapeutic needs and user preferences, as would be understood by those skilled in the art.

The heating pad 60 is equipped with a top cap 68 that houses essential circuitry as would typically be found to power and control an electric heating pad. This includes the circuitry required to drive the heating element, a battery for power, battery charging circuitry (the battery is charged through charge port 61 which, in one embodiment, is a USB-C connector), and control circuitry to regulate the heat. The control circuitry allows for temperature adjustments and ensures the pad maintains the desired temperature. The top cap 68 is designed to integrate seamlessly with the rest of the pad, ensuring both functionality and ease of use.

The construction of the heating pad 60 includes typical components found in modern heating pads, such as a flexible heating film embedded within the pad's layers, powered by the battery contained in the top cap. The control circuitry includes temperature sensors and a microcontroller to adjust the heat output based on user settings.

The secure fit provided by the rigid frame 14 and the careful design of the heating pad 60 ensure that the pad delivers consistent therapeutic heat, improving the overall effectiveness of the fertility treatment process.

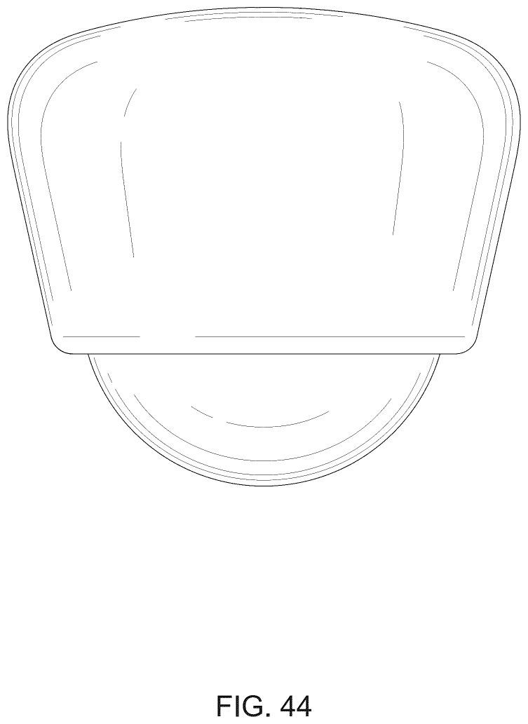

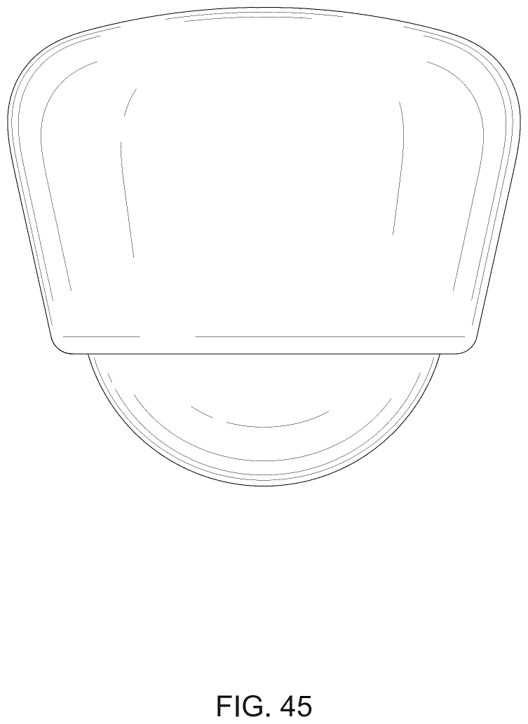

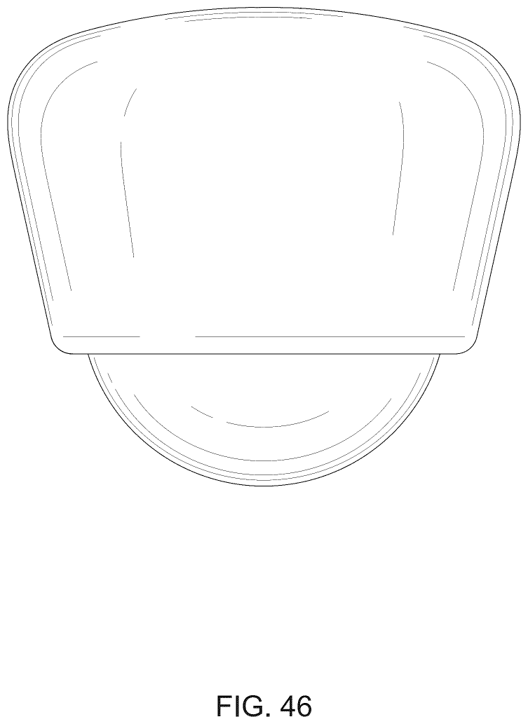

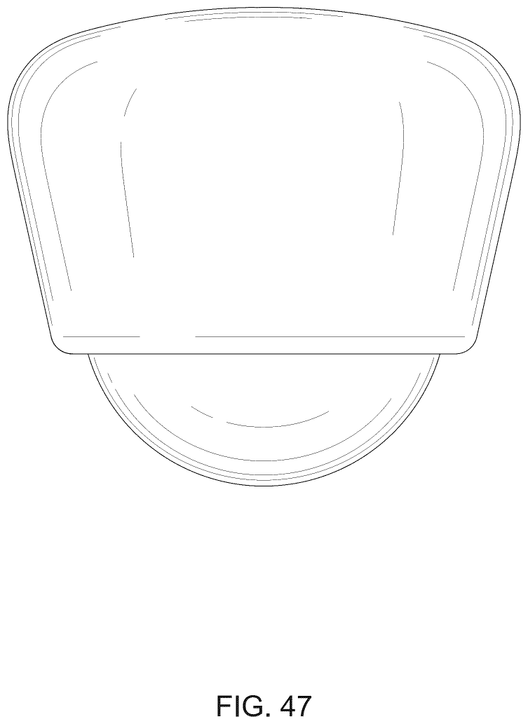

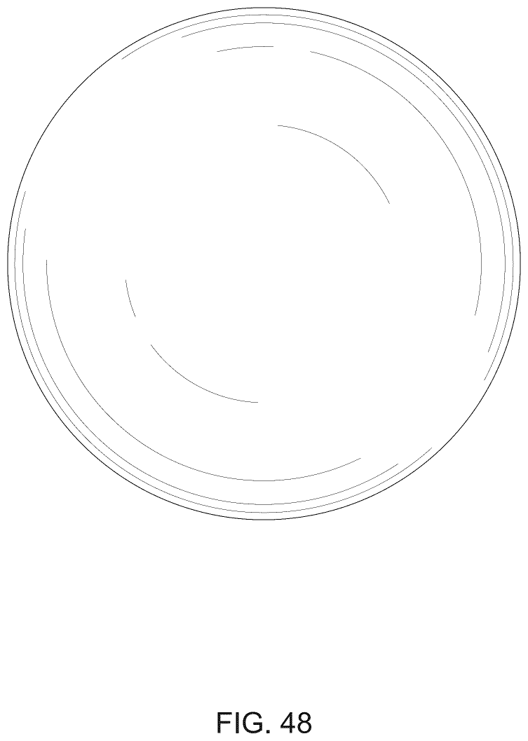

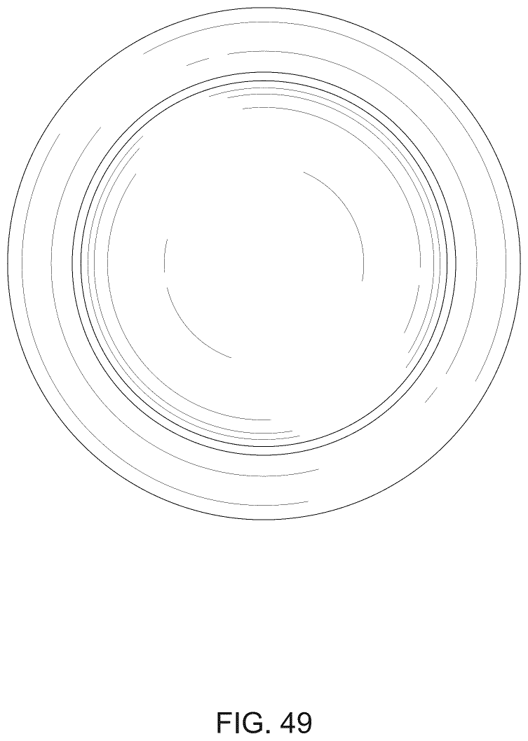

Turning now to FIGS. 42-49, the Fertility Treatment Compression Belt Kit further includes a massage ball 70. The massage ball is a handheld device designed to be used at the injection site after the administration of progesterone. Its primary function is to reduce “knots” that form under the skin due to the consolidation of progesterone in oil, and to improve the absorption of the progesterone once injected. In one embodiment, after the injection, the heating pad is applied to provide soothing relief, followed by the use of the massage ball to massage the injection site, enhancing comfort and therapeutic efficacy.

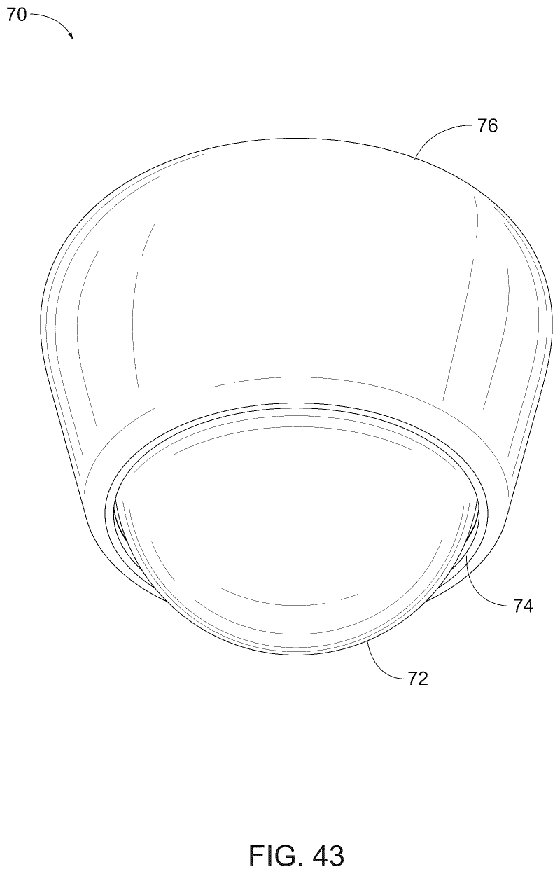

Referring to FIG. 43, massage ball 70 comprises a plastic ball 72 housed and retained within a plastic ball socket housing 74. Other types of materials may be used for ball 72, such as hard rubber. The ball socket housing 74 encases the ball and allows it to be held withing housing grip 76. Ball socket housing 74 ensures the ball remains securely in place while rotating and gliding smoothly during massaging action, allowing for effective and gentle massage of the injection site. The ball socket housing 74 is then inserted into a housing grip 76, which is ergonomically shaped to fit comfortably in the user's hand. The housing grip 76 is made of plastic and may include a coating or additional bonded layer, such as a silicon or textured rubber coating, to provide a softer, more comfortable surface that is easy to grip and manipulate.

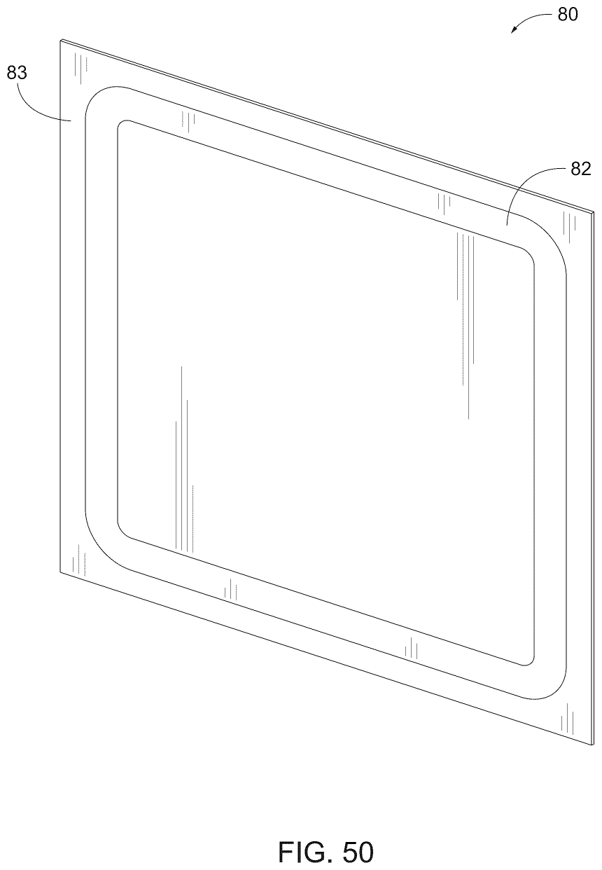

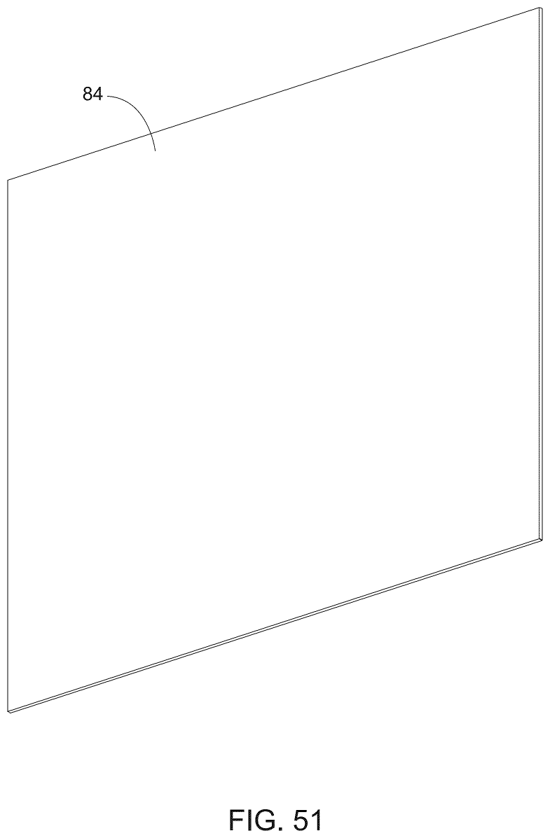

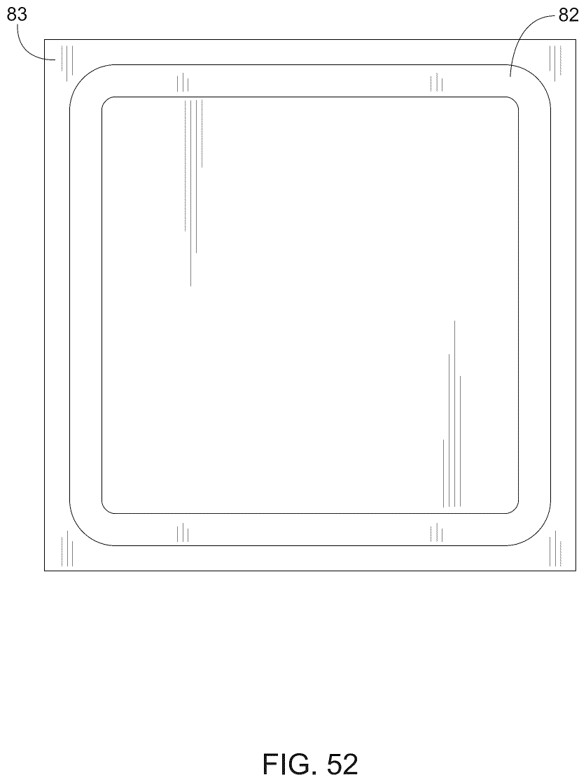

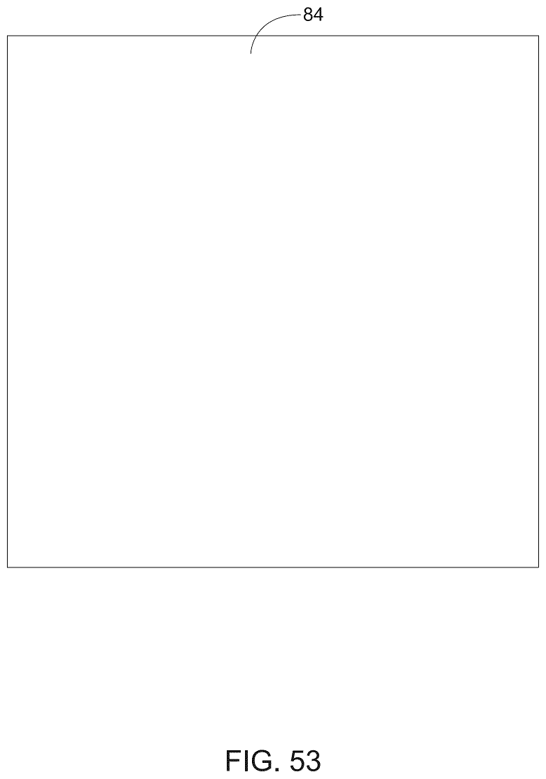

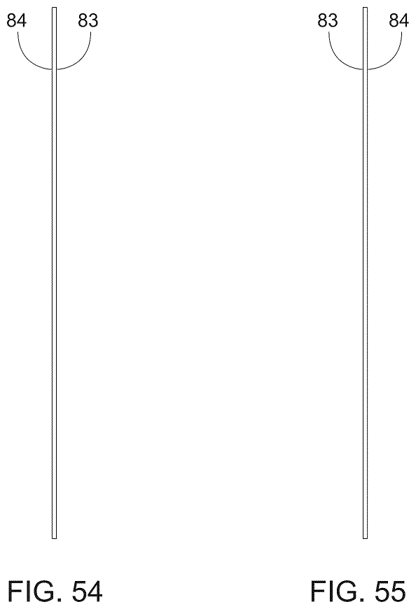

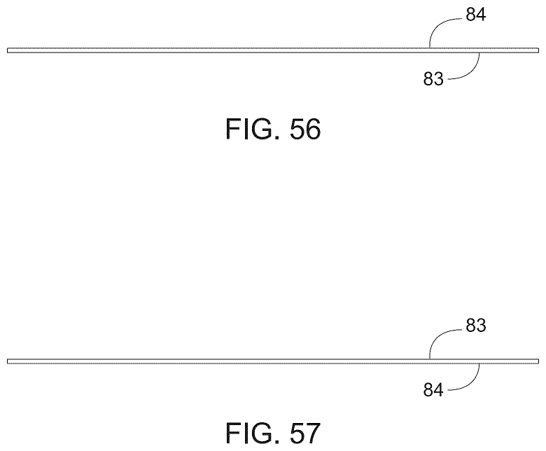

Turning now to FIGS. 50-57, the Fertility Treatment Compression Belt Kit further includes a Temporary Tattoo Injection Site Shot Target 80 is shown. Temporary tattoos are non-permanent designs that are applied to the skin. They are more generally used for decorative, promotional, and entertainment purposes. The construction of temporary tattoos are well understood by those having ordinary skill in the art and generally include a backing paper (84), an ink design layer with adhesive (82), and a clear top sheet transfer film 83 covering the ink design layer (82). To apply the temporary tattoo, the clear top sheet is removed, the clear top sheet 83 carrying design 82 is placed facing down against the skin, a wet cloth is placed against the clear top sheet 83 for approximately 30-60 seconds, and top sheet 83 is slowly peeled away from the skin leaving design 82 on the skin of the use.

The temporary tattoo 80 is applied to the skin at the injection site. It remains on the user for approximately a week. As it begins to fade, additional tattoos 80 may be applied.

Temporary tattoo 80 is sized so it is visible though cutout 12 when belt 10 is worn. Temporary tattoo 80 comprises a target indicator 82 which provides a visual mark on the user's skin to more accurately show where the injection should be made. In one embodiment target indicator 82 is a 2″×2″ rounded square 82 having a border of 3/16″ which is colored black. The inside area and outside areas of target indicator 82 are clear. It is appreciated that this is just one design for a temporary tattoo 80 and that target indicator 82 may include any number of designs, colors, and shades that provide an indication on the user's skin to accurately align the belt and indicate the precise location to administer the injection.

Additionally, since the tattoo 80 is semi-permanent, it provides a visual indication to the user showing where belt 10 should be positioned and aligned for subsequent IVF treatments. The user knows that belt 10 is properly positioned when target indicator 82 is visible through and approximately centered on cutout 12 of the belt.

In one embodiment, target 80 is applied to the upper left or upper right buttock of a user prior to intramuscular injection. In another embodiment, target 80 is applied to the abdomen region of the user prior to subcutaneous injection.

The tattoo removes any uncertainty about where to inject medication and ensures proper alignment of the belt each and every time. The various steps of the IVF process may be properly and comfortably completed while wearing the belt. In one embodiment, these steps include, putting belt 10 on so the target tattoo 80 is visible through cutout 12 of belt 10, inserting the cooling pad 50 into cutout 12 of belt 10 to numb the skin at the injection site, warming the injection vial in pocket 66 of heating pad 60 while the skin is being numbed, removing cooling pad 50 to expose the injection site, injecting the medication within the space outlined by target 82, inserting heating pad 60 into cutout 12 of belt 10 to provide comforting warmth to the skin at the injection site. By following these steps, the user can ensure accurate and consistent administration of the medication, enhancing both the comfort and effectiveness of the treatment process.

Various methods of use for the Fertility Treatment Compression Belt Kit have been disclosed above. These methods were discussed in connection with the detailed descriptions of the various parts of the kit, including their assembly and interaction. The following section will provide further information on the application and use of the belt, including the heating and cooling pads, injections, temporary tattoos, and massage ball. This comprehensive overview will ensure a clear understanding of how the components work together to enhance the fertility treatment process.

To apply the belt (10), the user starts with the window (accessory cutout 12) aligned to her back side and the hook and loop fastener facet out (away from her). The left side (13) of the belt is folded across the abdomen. The right side (11) is folded across the abdomen to connect the two sides (11, 13). The belt is tightened to achieve a snug fit by loosening the hook and loop faster connection and pulling each end (11, 13) of the belt in opposite directions. Once snug, the hook and loop fasteners are resecured. The belt is rotated so that the window (12) is aligned with the injection site. As disclosed above, if a temporary tattoo (80) has been applied the belt can be rotated so the window (12) is aligned with the temporary tattoo (80), thereby ensuring proper placement.

The injection site is typically either (a) the abdomen such as approximately one inch from either side of the belly button during injections to prepare for the egg removal (also called ovum retrieval) process which are subcutaneous injections (under the skin), or (b) the lower hip such as the upper buttock area for embryo transfer process with progesterone injections which are intramuscular injections. However, the exact protocol, types of injections, injection sites, and timing are determined by a fertility specialist.

### Egg Removal (Ovum Retrieval) Process With Subcutaneous Injections

Belt (10). The Belt is applied as disclosed above.

Cooling pad (50). To use the cooling pad (50), the cooling pad is placed in a freezer until it is completely frozen. When ready to use, the belt is positioned to the window (12) is aligned to the desired injection site (which may be indicated by the temporary tattoo (80) as disclosed above). The frozen cooling pad (50) is placed into the belt (10) from the bottom side (7) through the window (12) so the cap (58) of the cooling pad extends out through the front (9) of the belt and the bottom of pad (56) is flush against the skin of the user. The user should ensure that the belt is tight, and the pad is compressing against her skin. In one embodiment, the cooling pad is applied for at least 5 minutes.

Injection. For subcutaneous injections which are given for the egg retrieval process, after numbing, the cooling pad (50) is removed from the belt (10). The skin is sterilized and the injection is administered directly through the belt window (12).

Heating pad (30). Heat therapy is used after egg retrieval to help symptoms of cramping or discomfort. The heating pad (30) is inserted into the belt from the bottom side (7) through the window (12) so the cap (68) of the heating pad extends out through the front (9) of the belt and the bottom of the pad (64) is flush against the skin of the user. The heating pad is inserted so that it is horizontally oriented across the abdomen area, that is the longer width dimension is positioned horizontally. In this way, the user can enjoy hands-free heat therapy.

### Embryo Transfer Process With Intramuscular Injections

Warm Progesterone. The heating pad is powered on by pressing the vial heat button (65). In progesterone vial is inserted into the heat pad pocket (66). In one embodiment the vial is warmed for approximately 10 minutes.

Apply Belt (10). The belt is applied as disclosed above but with the window (12) aligned with the injection site on the user's hip. If a temporary tattoo (80) has been applied to indicate the injection site, the window (12) is aligned with the temporary tattoo (80).

Cooling Pad. To use the cooling pad (50), the cooling pad is placed in a freezer until it is completely frozen. When ready to use, the belt is positioned to the window (12) is aligned to the desired injection site (which may be indicated by the temporary tattoo (80) as disclosed above). The frozen cooling pad (50) is placed into the belt (10) from the bottom side (7) through the window (12) so the cap (58) of the cooling pad extends out through the front (9) of the belt and the bottom of pad (56) is flush against the skin of the user. The user should ensure that the belt is tight, and the pad is compressing against her skin. In one embodiment, the cooling pad is applied for at least 5 minutes.

Injection. For intramuscular injections which are given for embryo transfer, after numbing, the cooling pad (50) is removed from the belt (10). Ensuring that the window is (12) is aligned with the shot target (82), the skin is sterilized and the injection is administered directly through the window.

Heating Pad (60). As above, the heating pad (60) is inserted into the belt so that pad sits firmly against the skin. The heating pad is vertically oriented, that is the longer width dimension is positioned vertically. The heating pad is powered on to the desired temperature setting by pressing heat button (63). In this way, the user can enjoy hands-free heat therapy for as long as she desires.

Massage Ball (70). After heat therapy is applied, the belt (10) is removed and the massage ball (70) is rolled for a few minutes around the injection site. This helps relieve “knots” and “lumps” formed by the progesterone oil and aids in the absorption of the medication.

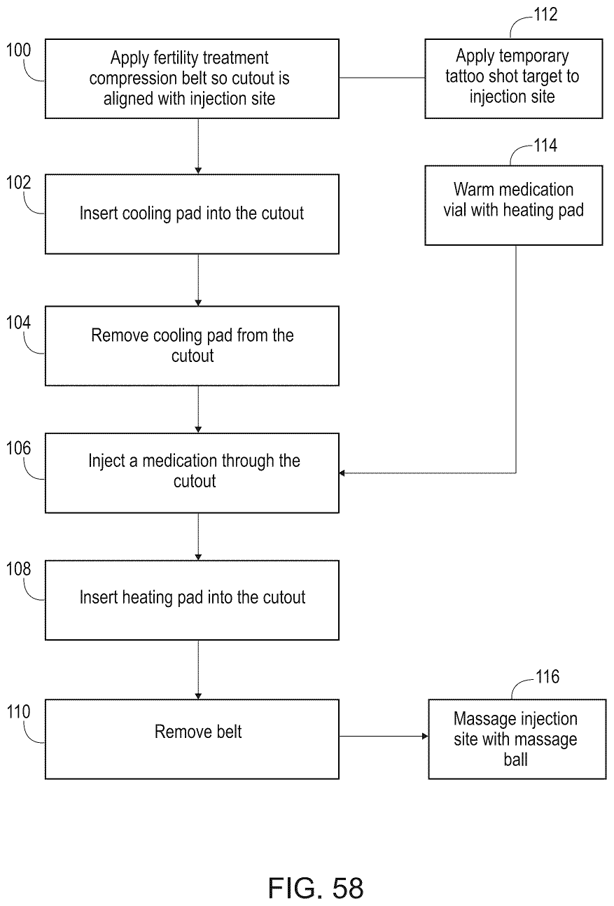

The methods disclosed above are illustrated in FIG. 58 which shows a method for performing fertility treatments. At step 100, the fertility treatment compression belt is applied so the cutout is aligned with the injection site. A temporary tattoo shot target may also have been applied to the injection site (step 112). The cooling pad is inserted into the cutout of the belt at step 102. After the injection site is numbed, the cooling pad is removed at step 104. At step 106, the medication is injected through the cutout. Depending on the procedure, the medication vial may be warmed with the heating pad in advance of the injection as indicated in step 114. After injecting the medication, the heating pad is inserted into the cutout at step 108. Depending on the procedure, after the area is soothed with the heating pad for a period of time determined by the user (for example 5-10 minutes), the belt is removed (and it follows that the heating pad is also removed) at step 110. Then, the injection site is massaged with the massage ball at step 116.

The foregoing detailed description has discussed only a few of the many forms that this invention can take. It is intended that the foregoing detailed description be understood as an illustration of selected forms that the invention can take and not as a definition of the invention. It is only the claims, including all equivalents, that are intended to define the scope of this invention.

What is claimed is:

1. A fertility treatment compression belt kit comprising:
a compression belt comprising a cutout, and having a top side, bottom side, left side, right side, back side, and front side;
a rigid frame affixed to the compression belt and surrounding the cutout;
a connector affixed to the right side of the belt; and
a connector affixed to the left side of the belt.

2. The kit of claim 1 wherein the compression belt is stretchable.

3. The kit of claim 1 wherein the cutout and rigid frame are in the shape of a rounded quadrilateral.

4. The kit of claim 1 further comprising:
an accessory having a top side, a bottom side, and a top cap affixed to the top side;
wherein the top side and bottom side of the accessory are larger than the cutout;
wherein the top cap extends from the top side of the accessory;
wherein the top cap is congruent to the cutout; and
wherein, when the accessory is inserted in the compression belt, the top cap extends upwardly from the bottom side of the belt, through the cutout, out through the top side of the compression belt.

5. The kit of claim 4 wherein the accessory is a heating pad.

6. The kit of claim 5 wherein the heating pad is a battery powered electric heating pad with at least two electronically controlled heat settings.

7. The kit of claim 5 wherein the bottom side of the heating pad comprises a pocket.

8. The kit of claim 4 wherein the accessory is a cooling pad.

9. The kit of claim 4 wherein the cutout, frame, and plane of the top cap is a rounded quadrilateral.

10. The kit of claim 1 further comprising a temporary tattoo injection site shot target, wherein the shot target is smaller than the size of the cutout.

11. The kit of claim 1 wherein the connector on the right side and the connector on the left side comprises a hook and loop fastener.

12. The kit of claim 1 where the compression belt comprises;
a foam core;
a top fabric and a bottom fabric surrounding and affixed to the foam core; and
wherein the rigid frame is located and affixed between the top fabric and bottom fabric.

13. The kit of claim 12 wherein the foam core, the top fabric, the bottom fabric, rigid frame, the right side connector, and the left side connector are affixed with a heat-pressed boding adhesive.

14. The kit of claim 1 further comprising a belt extender.

15. The kit of claim 1 further comprising a massage ball.

16. A fertility treatment compression belt kit comprising:
a compression belt comprising a cutout, and having a top side, bottom side, left side, right side, back side, and front side
a connector affixed to the right side of the belt;
a connector affixed to the left side of the belt; and
an accessory having a top side and a bottom side;
wherein the top side and bottom side of the accessory are larger than the cutout;
wherein the top side of the accessory has a top cap that is congruent to the cutout; and
wherein the top cap extends upwardly from the bottom side of the belt, through the cutout, out through the top side of the compression belt.

17. The fertility treatment compression belt kit of claim 16 further comprising:
a rigid frame affixed to the compression belt and surrounding the cutout;
wherein the rigid frame surrounds the central raised area of the accessory.

18. The fertility treatment compression belt kit of claim 16 wherein the accessory is a heating pad or a cooling pad.

19. The fertility treatment compression belt kit of claim 16 further comprising a belt extender.

20. The fertility treatment compression belt kit of claim 16 further comprising a temporary tattoo injection site shot target, wherein the shot target is smaller than the size of the cutout.

21. The fertility treatment compression belt kit of claim 16 further comprising a massage ball.

22. A method for performing fertility treatments comprising the steps of:
(a) applying a fertility treatment compression belt so a cutout of the belt is aligned with an injection site of a user;
(b) inserting a cooling pad into the cutout to numb the injection site;
(c) removing the cooling pad from the cutout when the injection site is numb;
(d) injecting a medication through the cutout; and
(e) inserting a heating pad into the cutout to soothe the injection site.

23. The method of claim 22 further comprising:
(a) applying a temporary tattoo shot target to the injection site; and
(b) aligning the cutout with the temporary tattoo.

24. The method of claim 22 further comprising, prior to injecting the medication, warming a medication vial with the heating pad.

25. The method of claim 24 where in the medication is warmed to approximately 99.6 degrees Fahrenheit.

26. The method of claim 22 further comprising:
(a) removing the heating pad and the belt; and
(b) massaging the injection site with a massage ball.

27. The method of claim 22 wherein the cooling pad is removed in (c) approximately five minutes or more after it is inserted in (b).

28. The method of claim 22 wherein the injection site is the user's abdomen.

29. The method of claim 22 wherein the injection site is the user's hip.
